(19) United States
(12) Patent Application Publication
(10) Pub. No.: US 2021/0315518 A1
(43) Pub. Date: Oct. 14, 2021

(54) POLYSOMNOGRAPHY MONITORING CAP

(71) Applicant: Brain Homecare Electronic Technology Co., Ltd., Shandong Province (CN)

(72) Inventors: Fazhan Gao, Shandong Province (CN); Wei-Lung Chiang, Shandong Province (CN); Peiyan Yu, Shandong Province (CN)

(21) Appl. No.: 17/111,171

(22) Filed: Dec. 3, 2020

(30) Foreign Application Priority Data
Apr. 14, 2020 (CN) 202010302735.5

(51) Int. Cl. A61B 5/00; A61B 5/291; A61B 5/296; A61B 5/297

(52) U.S. Cl. CPC A61B 5/4806; A61B 5/6803; A61B 5/291; A61B 5/296; A61B 5/297; A61B 2562/164; A61B 2562/0215; A61B 2562/046; A61B 2562/06; A61B 2562/222; A61B 2562/227; A61B 5/4815

(57) ABSTRACT

The present disclosure relates to a polysomnography monitoring cap, including a cap body and a plurality of electrodes preset on the cap body, where the cap body includes a head cover sheet for covering a head and a chin cover sheet for covering a mandible, and the head cover sheet and the chin cover sheet are connected into an integral cover sheet; the electrodes include a scalp electrode, an eye electrode and a mandible muscle electrode, where the scalp electrode is preset on the head cover sheet according to the international EEG positioning standard, the mandible muscle electrode is preset on the chin cover sheet, and the eye electrode is hung on the cap by a lead wire. According to the present disclosure, a scalp electrode, a mandible muscle electrode and an eye electrode are preset.

### CROSS REFERENCE TO RELATED APPLICATIONS

The application claims priority to Chinese Application No. 202010302735.5, filed on Apr. 14, 2020, entitled “Polysomnography Monitoring Cap,” which is hereby incorporated by reference in its entirety.

### TECHNICAL FIELD

The present disclosure relates to the technical field of polysomnography monitoring equipment, and in particular, to a polysomnography monitoring cap.

### BACKGROUND

Polysomnography (PSG) refers to the continuous and synchronous tracing of more than 10 indexes such as electroencephalograph (EEG) and respiration during overnight sleep. Monitoring and analysis of the sleep structure, progress, and abnormal EEG is one of the important examinations. Sleep analysis is usually performed by recording an electroencephalogram, an electrooculogram and an electromyogram to accurately reflect sleep status and stages. The electroencephalogram is used to distinguish sleep from awakening, and tell different sleep stages and the proportion of each sleep stage. The electrooculogram is used to observe the movement of an eyeball. The electromyogram is used to record electrical activity produced by muscle activity in the lower jaw region. Through sleep recording and analysis, sleep latency, awakening times and time, the proportion of sleeping, the proportion of awakening, the proportion of sleep stages, wake-up time and the total sleep duration can be known. Based on such information, the causes of some insomnia, such as depression and sleep respiratory disturbance can be evaluated and found. The quality of polysomnogram monitoring depends on the quality of electrode placement. Three key indexes of a sleep stage are electroencephalogram, electrooculogram and mandibular electromyogram.

The existing EEG cap is basically set as a scalp electrode cap which is only equipped with scalp electrodes according to the international 10-20 EEG standard and can collect only EEG. If the electrooculogram and the mandibular electromyogram need to be collected, the eye electrode and the myoelectric electrode need to be placed. During monitoring, it is necessary to measure, position, and install the eye electrode and the myoelectric electrode one by one, which poses high expertise requirements on monitoring personnel, takes time, and is limited by the monitoring requirements. In addition, most of the EEG electrodes currently used for monitoring are metal-plated electrodes. The electrodes are hard, which affects the contact between the electrodes and the scalp, and the long monitoring time will cause pressure pain at the contact position and discomfort in use. Therefore, it is necessary to design a polysomnography cap for sleep monitoring to record an electroencephalogram, an electrooculogram and an electromyogram simultaneously.

### SUMMARY

To resolve the defects of the prior art, the present disclosure provides a polysomnography monitoring cap that can realize simultaneous recording of an electroencephalogram, an electrooculogram and an electromyogram, also wear comfortable.

In order to achieve the above objective, the present disclosure provides a polysomnography monitoring cap, including a cap body and a plurality of electrodes preset on the cap body, where the cap body includes a head cover sheet for covering a head and a chin cover sheet for covering a mandible, and the head cover sheet and the chin cover sheet are connected into an integral cover sheet;

the electrodes include a scalp electrode, an eye electrode and a mandible muscle electrode, where the scalp electrode is preset on the head cover sheet according to the international EEG positioning standard, the mandible muscle electrode is preset on the chin cover sheet, and the eye electrode is hung on the cap by a lead wire.

Preferably, the scalp electrodes are set according to the international 10-20 EEG positioning standard, including frontal region electrodes F3 and F4, central region electrodes C3 and C4, occipital region electrodes O1 and O2, left and right mastoid reference electrodes M1 and M2, and a ground electrode GND.

Preferably, the eye electrode includes a right eye outer electrode EOG2 and a left eye outer electrode EOG1.

Preferably, each electrode uses copper foil as a base material, and a conductive layer is plated on the copper foil and connected with an electrode lead wire.

Preferably, the conductive layer is gold plated or silver chloride plated.

Preferably, each electrode is padded with a flexible gasket at the bottom, the flexible gasket is hollowed out in the centre, and a size of the hollow matches a size of the soft electrode.

Preferably, the electrode and the flexible gasket are covered by a flexible fabric, and the flexible fabric, the electrode and the flexible gasket are bonded by an adhesive sealant.

Preferably, the cap body is preset with a plurality of lead wire organizers, and each electrode is connected to a lead wire and then the lead wires are led out through the lead wire organizers.

Preferably, the polysomnography monitoring cap is further provided with a connector, and the lead wires connecting the electrodes are bound into a combined wire harness by the lead wire organizer and then connected to the connector.

Preferably, the joint between head cover sheet and the chin cover sheet are connected by a velcro.

Compared with the prior art, the present disclosure has the advantages and positive effects as follows.

The present disclosure provides a polysomnography monitoring cap, where a cap body is set as a head cover sheet and a chin cover sheet, a scalp electrode and a mandible muscle electrode are respectively preset on the head cover sheet and the chin cover sheet of the monitoring cap through preset electrodes, and an eye electrode is hung on the cap by a lead wire. When wearing of the polysomnography monitoring cap, it is only required to cover a head with the head cover sheet, cover a mandible with the chin cover sheet, and place the eye electrodes on the left eye and the right eye. There is no need to locate the electrodes one by one when using, and the operation is simple and convenient without requiring measurement and positioning, the difficulty of sleep monitoring is reduced, the use is convenient, and the electroencephalogram, the electrooculogram and the electromyogram are simultaneously recorded, to monitor the sleep quality. In addition, the traditional hard electrodes are replaced with the comfortable flexible soft electrodes, avoiding any scratching during wearing.

### BRIEF DESCRIPTION OF THE DRAWINGS

FIG. 1 is a structural view of a polysomnography monitoring cap of the present disclosure;

FIG. 2 is a structural view of electrode connection;

FIG. 3 is a structural view of a polysomnography monitoring cap with a head cover sheet and a chin cover sheet adhesively connected according to an embodiment 2 of the present disclosure; and

FIG. 4 is an enlarged view showing an adhesive connection structure between a head cover sheet and a chin cover sheet according to an embodiment 2 of the present disclosure.

In the figure: a cap body 1, a head cover sheet 11, a chin cover sheet 12, a lead wire organizer 13; an electrode 2, a scalp electrode 21, an eye electrode 22, a mandible muscle electrode 23; a lead wire 3, a connector 4, a flexible gasket 5, a flexible fabric 6, an adhesive sealant 7; a vertical connecting structure 8, a transverse connecting structure 9.

### DETAILED DESCRIPTION

In order to enable those skilled in the art to better understand the solutions of the present disclosure, the technical solutions in the embodiments of the present disclosure will be clearly described below in conjunction with the accompanying drawings in the embodiments of the present disclosure. Obviously, the described embodiments are some rather than all of the embodiments of the present disclosure. All other embodiments obtained by persons of ordinary skill in the art based on the embodiments of the present disclosure without creative efforts shall fall within the protection scope of the present disclosure.

Terms “include” and any variants thereof in the specification and claims of the present disclosure and in the above drawings are intended to cover a non-exclusive inclusion. For example, a process, method, system, product, or equipment that includes a list of steps or units is not limited to the listed steps or units, but may optionally further include steps or units not listed, or may optionally further include other steps or units inherent to such process, method, product, or equipment. Furthermore, the terms “first”, “second”, and “third”, and the like, are used for distinguishing between different objects and not for describing a particular order.

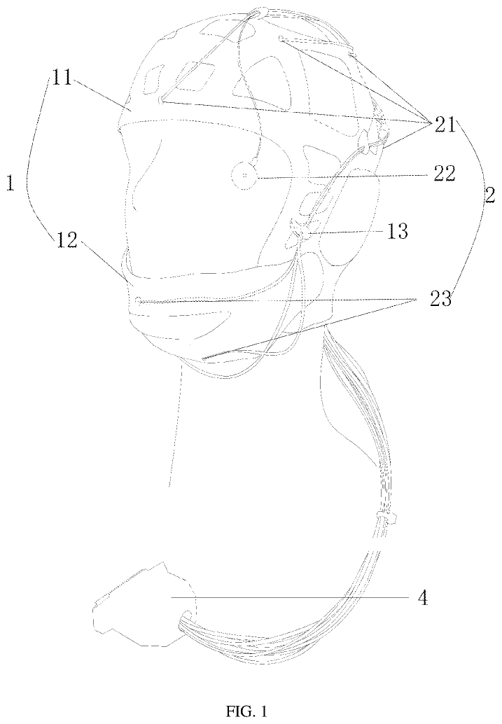

Referring to FIG. 1, an embodiment 1 of the present disclosure provides a polysomnography monitoring cap, including a cap body 1 and a plurality of electrodes 2 preset on the cap body, where the cap body 1 includes a head cover sheet 11 for covering a head and a chin cover sheet 12 for covering a mandible. In this embodiment, the head cover sheet 11 and the chin cover sheet 12 may be made of elastic materials and are arranged as a meshed one-piece structure. The head cover sheet 11 and the chin cover sheet 12 are connected into an integral cover sheet to form an elastic meshed cap body. Its structure is simple, and it is suitable for different people to wear.

As for the electrode 2 of the polysomnography monitoring cap, three types of electrodes, namely a scalp electrode 21, an eye electrode 22 and a mandible muscle electrode 23, are adopted, and each electrode is configured according to the recommended position of the American Academy of Sleep Medicine (AASM). The scalp electrode 21 is preset on the head cover sheet 11 according to the international electroencephalogram positioning standard, the mandible muscle electrode 23 is preset on the chin cover sheet 12, and the eye electrode 22 is hung on the cap by a lead wire. In addition, a plurality of lead wire organizers 13 are arranged on the cap body 1, the scalp electrode 21, the eye electrode 22 and the mandible muscle electrode 23 each are connected to one lead wire 3, and then the lead wires 3 are led out through a lead wire organizer 13 preset on the monitoring cap, are bound into a combined wire harness through the lead wire organizer and then are connected to a connector 4. The connector 4 is connected to electroencephalogram detection equipment to transmit a brain wave signal, an electro-ocular wave signal and a mandible myoelectric wave signal collected by the electrodes to the electroencephalogram detection equipment, so as to record an electroencephalogram, an electrooculogram and an electromyogram simultaneously and monitor human sleep quality.

The scalp electrode in this embodiment may be specifically set according to the international 10-20 system of EEG electrode placement, with a relative position between scalp electrode points expressed by 10% and 20%, including frontal region electrodes F3 and F4, central region electrodes C3 and C4, occipital region electrodes O1 and O2, left and right mastoid reference electrodes M1 and M2, and a ground electrode GND. The eye electrode includes a right eye outer electrode EOG2 and a left eye outer electrode EOG1. In this embodiment, the scalp electrode is set according to the 10-20 standard, but the number of the electrodes is not limited to this in a specific design, and can be further extended based on this embodiment according to the 10-20 or 10-10 standard, for example, 16 leads, 32 leads, or 64 leads.

According to this embodiment, a preset electrode mode is adopted, where the scalp electrode 21 and the mandible muscle electrode 23 are respectively preset on the head cover sheet 11 and the chin cover sheet 12 of the monitoring cap, and the eye electrode 22 is hung on the cap by a lead wire, so that the placement position of the eye electrode can be adjusted according to different wearers. In this way, no positioning is needed during use, eliminating the need of measurement and positioning, reducing the difficulty of sleep monitoring and facilitating the use. In addition, the electrodes 2 are connected to the lead wires 3 and then fastened on the cap body 1 through the lead wire organizer 13. The lead wires are bound into a combined wire harness through the lead wire organizer and then connected to the connector 4. The connector 4 can be connected to electroencephalogram monitoring equipment. The use of the lead wire organizer allows the lead wires to be smoothly organized, avoiding signal interference caused by disordered lead wires, preventing each lead wire from falling off from the electrode or being damaged, and facilitating the connection with the connector 4. Preferably, the connector 4 is an HDMI connector.

When wearing, the head cover sheet is coated on the head of a human body, the chin cover sheet is coated on the mandible, and the eye electrodes are arranged on the left eye and the right eye. Information of each electrode of the brain of a human body is collected through each scalp electrode to generate an electroencephalogram, so as to judge the sleep state of the human body. Electrode information of the left and right eyes of a human body is collected through two eye electrodes to generate an electrooculogram, so as to observe the movement state of the eyeballs. In addition, electrode information of the mandible part of the human body is collected through the mandible muscle electrode to generate a mandibular electromyogram, so as to observe the muscle activity state of the lower jaw part. In this way, the electroencephalogram, electrooculogram and electromyogram are recorded simultaneously, to monitor the sleep quality of the human body.

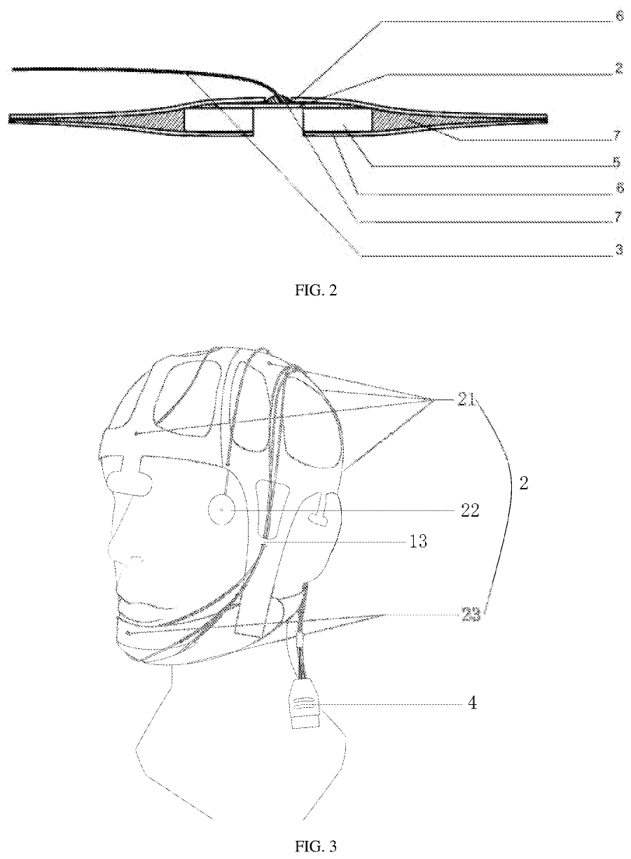

In addition, in this embodiment, each electrode is a flexible electrode that uses copper foil as a base material, and a conductive layer is plated on the copper foil and connected with an electrode lead wire. The conductive layer may specifically adopt gold plating or silver chloride plating which is good in conductivity and low in noise, polarization potential and impedance. As shown in FIG. 2, each electrode 2 is padded with a flexible gasket 5 at the bottom, which is designed as a middle hollow-out structure according to the size of the electrodes to facilitate the replacement of the electrodes. In addition, the electrode 2 and the flexible gasket 5 are coated by a flexible fabric 6. The flexible fabric 6, the electrode 2 and the flexible gasket 5 are bonded by an adhesive sealant 7, to improve the sealing effect of the electrodes, prolong the service life and increase the monitoring precision. Due to the use of the flexible gasket and the flexible fabric, the pressure pain caused by the traditional hard electrode is reduced and the feeling of comfort during sleep monitoring is enhanced. A gap for connecting the lead wires 3 is reserved in the flexible fabric 6 coated on the surface layer of the electrode 2, and the lead wire 3 is attached to the surface layer of the electrode 2 through the adhesive sealant 7, so as to facilitate the replacement of the lead wire.

The present disclosure also provides an embodiment 2 of the polysomnography monitoring cap in which the head cover sheet 11 and the chin cover sheet 12 are adhesively connected.

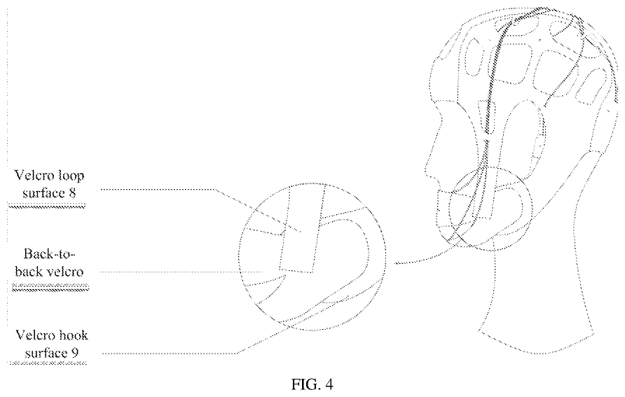

As shown in FIGS. 3 and 4, on both sides of the cap body 1, the joint between the head cover sheet 11 and the chin cover sheet 12 is connected by a velcro, and the adhesive connection structure and the adhesive mode are the same. The head cover sheet 11 includes a vertical connecting structure 8 and a transverse connecting structure 9 at the joint of the head cover sheet 11 and the chin cover sheet 12, a Velcro loop surface is arranged on the outer side of the vertical connecting structure 8, a velcro hook surface is arranged on the transverse connecting structure 9, and a back-to-back velcro is arranged on the chin cover sheet 12.

When wearing, the head cover sheet 11 is coated around the head of the human body, and the chin cover sheet 12 is coated around the mandible. The loop surface of the velcro of the vertical connecting structure 8 of the head cover sheet 11 adheres to the hook surface of the back-to-back velcro of the chin cover sheet 12 to realize vertical fixation. The hook surface of the velcro of the transverse connecting structure 9 of the head cover sheet 11 adheres to the loop surface of the back-to-back velcro of the chin cover sheet 12 to realize transverse fixation.

The vertical connecting structure 8 and the transverse connecting structure 9 of the head cover sheet are respectively adhered to and fixed with the chin cover sheet, so that the vertical direction and the transverse direction of the cap body can be adjusted separately or adjusted together, to meet the wearing requirements of different head shapes of users, better fit the cap body to the head of the user, and make the user feel more comfortable when wearing the cap.

In summary, according to the polysomnography monitoring cap provided by the present disclosure, the electrodes are preset, where the scalp electrode 21 and the mandible muscle electrode 23 are respectively preset on the head cover sheet 11 and the chin cover sheet 12 of the monitoring cap, and the eye electrode 22 is hung on the cap by a lead wire. When wearing of the polysomnography monitoring cap, it is only required to cover the head with the head cover sheet 11, cover the mandible with the chin cover sheet, and place the eye electrodes 22 on the left eye and the right eye. There is no need to locate the electrodes one by one when using, and the operation is simple and convenient without requiring measurement and positioning, the difficulty of sleep monitoring is reduced, the use is convenient, and the electroencephalogram, the electrooculogram and the electromyogram are simultaneously recorded, to monitor the sleep quality of a human body. In addition, the traditional hard electrodes are replaced with the comfortable flexible soft electrodes, avoiding any scratching during wearing.

The above are only preferred embodiments of the present disclosure, and are not intended to limit the present disclosure in other forms. Those skilled in the art may change or modify the technical content disclosed above into equivalent embodiments of equivalent changes without departing from the technical solutions of the present disclosure. Any simple modifications, equivalent changes and modifications made to the above embodiments based on the technical essence of the present disclosure still belong to the protection scope of the technical solutions of the present disclosure.

1. A polysomnography monitoring cap, comprising a cap body and a plurality of electrodes preset on the cap body, wherein the cap body comprises a head cover sheet for covering a head and a chin cover sheet for covering a mandible, and the head cover sheet and the chin cover sheet are connected into an integral cover sheet; and
the electrode comprises a scalp electrode, an eye electrode and a mandible muscle electrode, wherein the scalp electrode is preset on the head cover sheet according to an international EEG positioning standard, the mandible muscle electrode is preset on the chin cover sheet, and the eye electrode is hung on the cap by a lead wire.

2. The polysomnography monitoring cap according to claim 1, wherein the scalp electrode is set according to the international 10-20 EEG positioning standard and comprises frontal region electrodes F3 and F4, central region electrodes C3 and C4, occipital region electrodes O1 and O2, left and right mastoid reference electrodes M1 and M2, and a ground electrode GND.

3. The polysomnography monitoring cap according to claim 1, wherein the eye electrode comprises a right eye outer electrode EOG2 and a left eye outer electrode EOG1.

4. The polysomnography monitoring cap according to claim 1, wherein each electrode is made of a copper foil base material, and is connected to a lead wire after being plated with a conductive layer via a copper foil base sheet.

5. The polysomnography monitoring cap according to claim 4, wherein the conductive layer is gold plated or silver chloride plated.

6. The polysomnography monitoring cap according to claim 4, wherein each electrode is padded with a flexible gasket at the bottom, the flexible gasket is hollowed out in the centre, and a size of the middle hollow matches a size of a soft electrode.

7. The polysomnography monitoring cap according to claim 6, wherein the electrode and the flexible gasket are covered by a flexible fabric, and the flexible fabric, the electrode and the flexible gasket are bound by an adhesive sealant.

8. The polysomnography monitoring cap according to claim 1, wherein the cap body is preset with a plurality of lead wire organizers, and each electrode is connected to a lead wire and then the lead wires are led out through the lead wire organizers.

9. The polysomnography monitoring cap according to claim 8, wherein a connector is further provided, and the lead wires connecting the electrodes are bound into a combined wire harness by the lead wire organizer and then connected to the connector.

10. The polysomnography monitoring cap according to claim 2, wherein each electrode uses copper foil as a base material, and a conductive layer is plated on the copper foil and connected with an electrode lead wire.

11. The polysomnography monitoring cap according to claim 3, wherein each electrode uses copper foil as a base material, and a conductive layer is plated on the copper foil and connected with an electrode lead wire.

12. The polysomnography monitoring cap according to claim 10, wherein the conductive layer is gold plated or silver chloride plated.

13. The polysomnography monitoring cap according to claim 11, wherein the conductive layer is gold plated or silver chloride plated.

14. The polysomnography monitoring cap according to claim 10, wherein each electrode is padded with a flexible gasket at the bottom, the flexible gasket is hollowed out in the centre, and a size of the middle hollow matches a size of a soft electrode.

15. The polysomnography monitoring cap according to claim 11, wherein each electrode is padded with a flexible gasket at the bottom, the flexible gasket is hollowed out in the centre, and a size of the middle hollow matches a size of a soft electrode.

16. The polysomnography monitoring cap according to claim 14, wherein the electrode and the flexible gasket are covered by a flexible fabric, and the flexible fabric, the electrode and the flexible gasket are bound by an adhesive sealant.

17. The polysomnography monitoring cap according to claim 15, wherein the electrode and the flexible gasket are covered by a flexible fabric, and the flexible fabric, the electrode and the flexible gasket are bound by an adhesive sealant.

18. The polysomnography monitoring cap according to claim 1, wherein the join between the head cover sheet and the chin cover sheet are connected by a velcro.
